(19) United States
(12) Patent Application Publication
(10) Pub. No.: US 2023/0041580 A1
(43) Pub. Date: Feb. 9, 2023

(54) SURGICAL INSTRUMENTATION EDUCATIONAL AND TRAINING PLATFORM

(71) Applicant: Ninja Solutions, Inc., Dix Hills, NY (US)

(72) Inventors: David I. Zaret, Dix Hills, NY (US); Eric Price, Dix Hills, NY (US)

(21) Appl. No.: 17/393,861

(22) Filed: Aug. 4, 2021

(51) Int. Cl. G09B 23/28; G16H 20/40

(52) U.S. Cl. CPC G09B 23/28; G16H 20/40

(57) ABSTRACT

A computer-implemented method for employing a surgical instrument educational platform to train medical personnel to identify, characterize, and organize surgical instrumentation and surgical instrumentation trays includes selecting a level from one or more levels on a display of an electronic device, selecting a surgical category from a plurality of surgical categories, in response to selecting the surgical category, an image of a surgical instrument is displayed on the display of the electronic device and a query is made to identify the image of the surgical instrument, loading surgical instrument options to allow a user to associate the image of the surgical instrument to one of the surgical instrument options, prompting the user to select an option from the surgical instrument options, and in response to the selected option by the user, outputting a result.

### BACKGROUND

The present invention relates generally to educational software systems, and more specifically, to a surgical instrumentation educational and training platform.

A number of teaching and learning systems have been developed over the years for assisting students or users in learning via software application programs on a computer. However, these conventional methods are considered to be deficient because they do not truly provide fully interactive computer systems which can provide a simplified, easy to use and fastest learning system possible. Moreover, gamification may be defined as using game elements in a non-gaming context to improve user motivation and experience. Studies have shown that gamification can have vast benefits. Adding game elements to teaching and learning systems can simulate primitive, competitive and collaborative instincts that are normally active in people.

It is with this object in mind that the present invention has been developed.

### SUMMARY

In accordance with an embodiment, a computer-implemented method for employing a surgical instrument educational platform to train medical personnel to identify, characterize, and organize surgical instrumentation and surgical instrumentation trays is provided. The computer-implemented method includes selecting a level from one or more levels on a display of an electronic device, selecting a surgical category from a plurality of surgical categories, in response to selecting the surgical category, an image of a surgical instrument is displayed on the display of the electronic device and a query is made to identify the image of the surgical instrument, loading surgical instrument options to allow a user to associate the image of the surgical instrument to one of the surgical instrument options, prompting the user to select an option from the surgical instrument options, and in response to the selected option by the user, outputting a result.

In accordance with another embodiment, a computer-implemented method for employing a surgical instrument educational platform to train medical personnel to identify, characterize, and organize surgical instrumentation and surgical instrumentation trays is provided. The computer-implemented method includes selecting a level from one or more levels on a display of an electronic device, selecting a surgical category from a plurality of surgical categories, in response to selecting the surgical category, displaying a plurality of surgical instruments moving on a conveyor belt and displaying at least one surgical instrumentation tray, prompting the user to select and drag any of the plurality of surgical instruments moving on the conveyor belt onto the at least one surgical instrumentation tray, and in response to positioning the plurality of surgical instruments moving on the conveyor belt onto the at least one surgical instrumentation tray, outputting a result.

In accordance with yet another embodiment, a computer-implemented method for employing a surgical instrument educational platform to train medical personnel to identify, characterize, and organize surgical instrumentation and surgical instrumentation trays is provided. The computer-implemented method includes selecting a level from one or more levels on a display of an electronic device, selecting a surgical category from a plurality of surgical categories, in response to selecting the surgical category, concurrently displaying all of a plurality of surgical instruments in a jumbled manner within a flashcard and displaying at least one surgical instrumentation tray, prompting the user to select and drag at least one surgical instrument of the plurality of surgical instruments onto the at least one surgical instrumentation tray, and in response to positioning the at least one surgical instruments of the plurality of surgical instruments onto the at least one surgical instrumentation tray, outputting a result.

It should be noted that the exemplary embodiments are described with reference to different subject-matters. In particular, some embodiments are described with reference to method type claims whereas other embodiments have been described with reference to apparatus type claims. However, a person skilled in the art will gather from the above and the following description that, unless otherwise notified, in addition to any combination of features belonging to one type of subject-matter, also any combination between features relating to different subject-matters, in particular, between features of the method type claims, and features of the apparatus type claims, is considered as to be described within this document.

These and other features and advantages will become apparent from the following detailed description of illustrative embodiments thereof, which is to be read in connection with the accompanying drawings.

### BRIEF DESCRIPTION OF THE DRAWINGS

The invention will provide details in the following description of preferred embodiments with reference to the following figures wherein:

FIG. 1 illustrates an opening screen of the surgical instrumentation training platform displayed on a display screen depicting a plurality of gates or levels, in accordance with an embodiment of the present invention;

FIG. 2 illustrates a screenshot of categories of surgical instruments, in accordance with an embodiment of the present invention;

FIG. 3 illustrates a screenshot depicting a surgical instrument after the user selects a category of surgical instruments in FIG. 2, where the user is prompted to identify the depicted surgical instrument, in accordance with an embodiment of the present invention;

FIG. 4 illustrates a screenshot where the user selects an answer to the question posed in FIG. 3, in accordance with an embodiment of the present invention;

FIG. 5A illustrates a screenshot where the user checks the answer provided to the question posed, in accordance with an embodiment of the present invention;

FIG. 5B illustrates the back of the flashcard after the check answer button has been tapped, in accordance with an embodiment of the present invention;

FIG. 6 illustrates a screenshot depicting another surgical instrument after the user identifies the previous surgical instrument, where the user is prompted to identify the depicted surgical instrument, in accordance with an embodiment of the present invention;

FIG. 7 illustrates a screenshot where the user has entered a second level after the first level has been successfully completed, the second level depicting surgical instruments on a conveyor belt and prompting the user to drag the surgical instruments into the correct surgical instrument category, in accordance with an embodiment of the present invention;

FIG. 8 illustrates a screenshot depicting a scorecard of the user, in accordance with an embodiment of the present invention;

FIG. 9 illustrates a screenshot where the user needs to assemble trays of instrumentation, in accordance with an embodiment of the present invention;

FIG. 10A illustrates a screenshot where a plurality of surgical instruments are presented and the user must assemble them into appropriate surgical categories, in accordance with an embodiment of the present invention;

FIG. 10B illustrates a screenshot of further magnified views of surgical instruments of a surgical tray, in accordance with an embodiments of the present invention;

FIG. 10C illustrates a screenshot of a single magnified instrument, in accordance with an embodiment of the present invention;

FIG. 11 illustrates a screenshot of the user selecting surgical instruments from the plurality of surgical instruments shown in FIG. 10A, in accordance with an embodiment of the present invention;

FIG. 12 illustrates a screenshot of the user dragging the selected surgical instruments onto a surgical instrument tray, in accordance with an embodiment of the present invention;

FIG. 13 illustrates a flowchart of how the user identifies, characterizes, and organizes surgical instrumentation and surgical instrumental trays of the surgical instrumentation training platform, in accordance with an embodiment of the present invention; and

FIG. 14 is a block/flow diagram of an exemplary processing system employed in a surgical instrument gamification environment, in accordance with an embodiment of the present invention.

Throughout the drawings, same or similar reference numerals represent the same or similar elements.

### DETAILED DESCRIPTION

Computer-based training, also known as technology-based training or e-learning, has many advantages over traditional, classroom-based learning environments. For example, students may typically take advantage of the training at a particular time or in a particular geographic location that is convenient to them. Students may each proceed through the training at their individual paces, and obtain virtually instantaneous feedback as they do so. Moreover, computer-based training has the potential to be extremely cost-effective, particularly for large corporations that must train their employees as their employees are hired or otherwise as needed.

Embodiments in accordance with the present invention provide for a training platform to teach or train operating room personnel about surgical equipment. The exemplary surgical instrumentation training platform teaches a user to identify, characterize, and organize surgical instrumentation and surgical instrumentation trays. The surgical instrumentation training platform can be used on mobile, tablet, laptop, or desktop or any other electronic device. The surgical instrumentation training platform can be in the form of a downloadable application and/or a web-based platform. Elements of gamification such as points, badges, streaks, and leaderboards are included in the surgical instrumentation training platform. Reports on user performance are also generated for educators and administrators (or mentors/teachers).

The present invention is directed generally to educational software. More particularly, the present invention is directed toward educational software for use in training operating room and sterile processing department personnel (or medical personnel). More specifically, the present invention is directed toward educational software for use in training operating room and sterile processing department personnel (or medical personnel) who need to learn to identify, characterize, and organize surgical instrumentation and surgical instrumentation trays. A computer-implemented method for employing a surgical instrument educational platform is presented, the computer-implemented method being implemented in a host computer having one or more physical processors programmed with computer program instructions that, when executed by the one or more physical processors, cause the host computer to perform the method of surgical instrumentation recognition and identification.

As will be discussed in detail in the description of the preferred embodiment which is set forth subsequently, the surgical instrumentation training platform in accordance with the present invention includes a methodology to teach or train the user to identify unique surgical devices and instruments. Once the user identifies a surgical instrument, the user also learns to categorize the surgical instrument according to its surgical instrument category, for example, a category of “retractors” vs “clamps,” and also categorize the surgical instrument according to its surgical type category. For example, a user can learn to differentiate between an instrument that is utilized in urology versus an instrument that is utilized in orthopedic surgery.

Further, the user is trained to assemble surgical trays of surgical instruments. For ease of explanation, surgical trays are used to organize, sterilize, and transport surgical instruments for use in a surgical operating room. There are two types of surgical trays. “Industry trays,” which are used to house a specific manufacturer's instruments. These trays are usually constructed such that each instrument occupies a very specific location in the tray. “Domestic trays,” which are usually created and customized by individual hospitals and are often a combination of surgical instruments from several different manufacturers. In most cases, the surgical instruments do not have specific locations on a surgical instrumentation tray, however, they are often loaded onto an instrument organizing device called a stringer, which is then placed into a surgical tray.

It is to be understood that the present invention will be described in terms of a given illustrative architecture; however, other architectures, structures, substrate materials and process features and steps/blocks can be varied within the scope of the present invention. It should be noted that certain features cannot be shown in all figures for the sake of clarity. This is not intended to be interpreted as a limitation of any particular embodiment, or illustration, or scope of the claims.

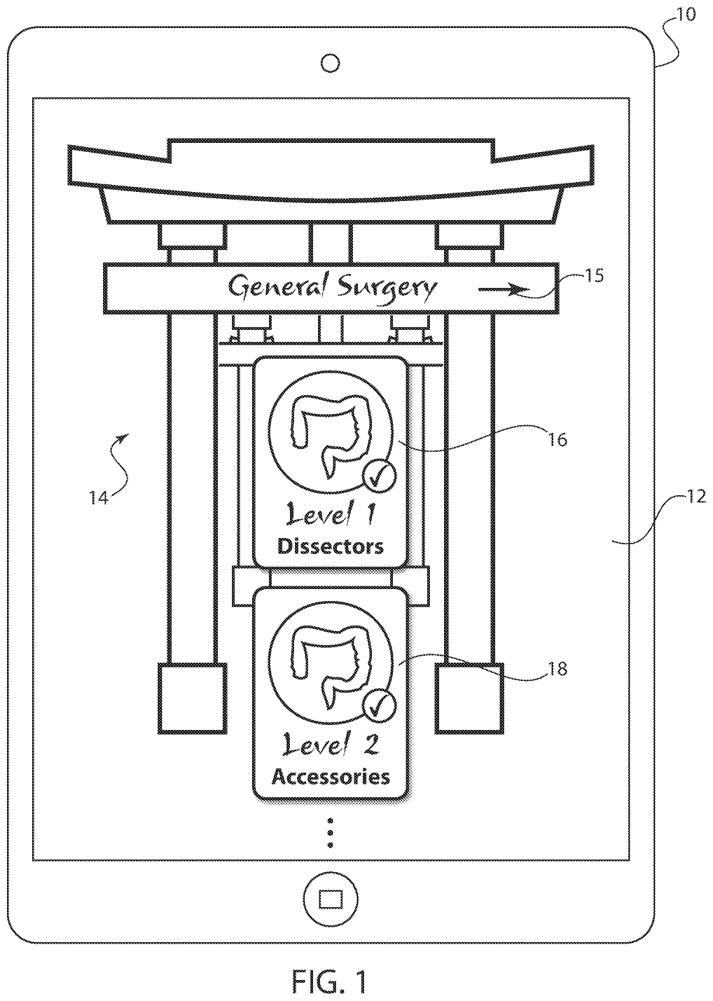

FIG. 1 illustrates an opening screen of the surgical instrumentation training platform displayed on a display screen depicting a plurality of gates or levels, in accordance with an embodiment of the present invention.

A user can employ an electronic device 10 having a display screen 12 to operate the surgical instrumentation training platform. The electronic device 10 can be, e.g., a mobile device, such as a smart phone or tablet or laptop. The electronic device 10 can be any device including a display screen 12, including a monitor or television screen. The user can also be referred to as a learner or a participant or a player.

The term “player,” as used herein, can refer to a person who interacts with an educational system for the purpose of gaining knowledge, insight, or understanding of one or more target surgical instruments or by virtue of having been allowed, asked, or assigned to interact with the educational training system.

As used herein, a “user” can be a learner or a player or a training manager, and is usually a person for learning e-learning courses and/or content within the invented system and method. The user can be, e.g., medical personnel that need to be trained for surgical instrumentation recognition and identification.

The term “lesson,” as used herein, can refer to one or more learning activities that teach toward a specific educational objective or a specific element of a published educational standard.

The term “level of study,” as used herein, can refer to one or more learning units that teach toward a plurality of general educational objectives or an overarching element of a published educational standard.

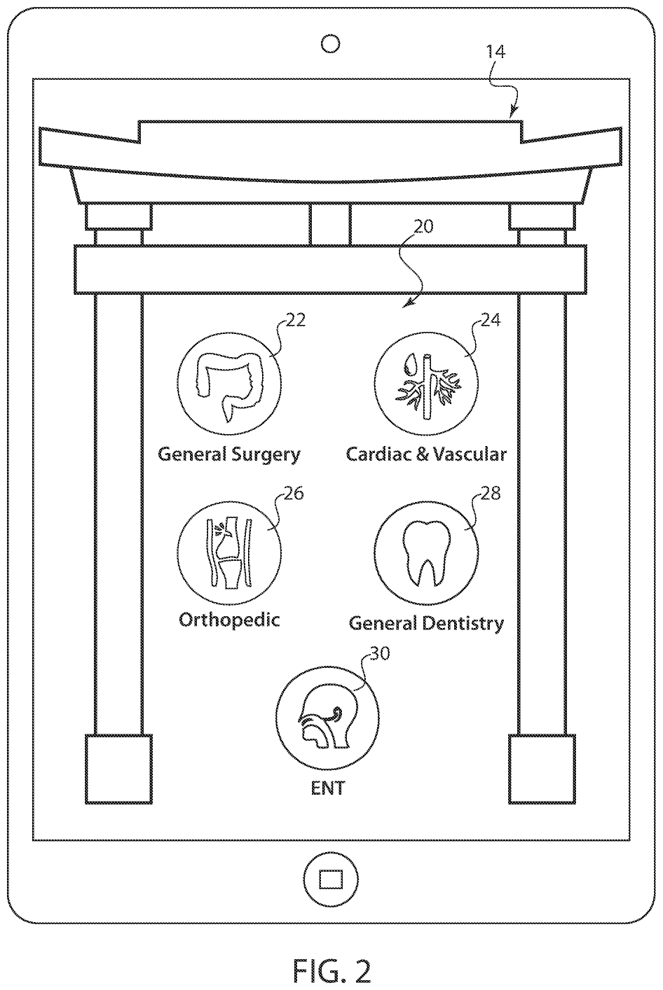

When the user opens the surgical instrumentation training platform on the electronic device 10, a display screen 12 shows the user a plurality of gates 14. Each gate of the plurality of gates 14 represents a level of play and proficiency. A user can select a gate 14 or level of play. Each level includes categories of surgical instrumentation, as described below with reference to FIG. 2. Each gate 14 can include a play button 15. The play button 15 allows the user to pass to the next level. In one example, a gate 14 can be labeled as “General Surgery.” The “General Surgery” gate 14 can include the play button 15 for allowing the user to move to the next level. Categories of instrumentation are provided within the gates 14. For example, a first category 16 and a second category 18 are illustrated within or underneath the gates 14. The first category 16 can be, e.g., “Dissectors,” whereas the second category 18 can be, e.g., “Accessories.” Each category 16, 18 can include a checkmark adjacent the icon to indicate task completion. When a task has been completed, the next task may be unlocked for access by the user. Additionally, each category 16, 18 may be assigned a level, such as “Level 1,” “Level 2,” etc. It is noted that FIG. 2 below provides another example with several more options for categories of surgical instrumentation. Each of those categories in FIG. 2 can also include at least checkmarks as well as level designations.

Typically, games include multiple levels and multiple stages, each level having a plurality of blocks and each stage having a plurality of levels. In a typical multilevel game, the game participant is required to achieve a certain threshold of performance in order to “unlock” another, more difficult level. Many of the games are also grouped into progressive (and optionally overlapping) tiers, requiring the game participant to satisfactorily complete all of the games in a given tier, and a sufficient number of levels within those games, before providing access to the games of the next tier.

In some embodiments, the games (or levels 14) increase in difficulty when the participant exceeds a threshold of success, and they decrease in difficulty when the participant's performance drops below another threshold. Many of the games enable a participant to “unlock” a new level merely by beating the participant's previous best score.

Furthermore, as used herein, display screen 12 refers to a display device such as a television or computer monitor (LCD, CRT, PDP, etc.), video billboard, video projector, LED display device (e.g., a screen that scrolls text using LEDs), and/or movie screen that normally operates to display non-gaming media content for viewing. A display screen 12 may be considered to be a display that is (or may be) simultaneously viewable by more than one person such as a projection of content onto a wall or other smooth surface. A display screen 12 typically, in normal operation, displays non-gaming media content for viewing by persons not associated with a particular game. A display screen 12 may have associated audio speakers (e.g., for outputting the sound of a video feed). In an exemplary embodiment, these audio speakers may be considered to be part of the display screen 12. In another exemplary embodiment, display screen 12 may be a portable display device or display surface.

FIG. 2 illustrates a screenshot of categories of surgical instruments, in accordance with an embodiment of the present invention.

A plurality of icons with instrumentation categories 20 is presented to the user. The plurality of icons are positioned within or underneath a gate 14. For example, if the user selected level 1, the following instrumentation categories 20 can be presented: general surgery 22, cardiac and vascular 24, orthopedic 26, general dentistry 28, and ear, nose, and throat (ENT) 30. Of course, one skilled in the art can contemplate a number of other surgical instrumentation categories to include. The surgical instruments can be categorized according to the types of surgical instruments, e.g., clamp instruments or retractor instruments, etc. The surgical instruments can be further categorized according to types of surgery, e.g., eye surgery, neurosurgery, heart surgery, etc. A user can select a surgical category as represented by an icon to begin training or play.

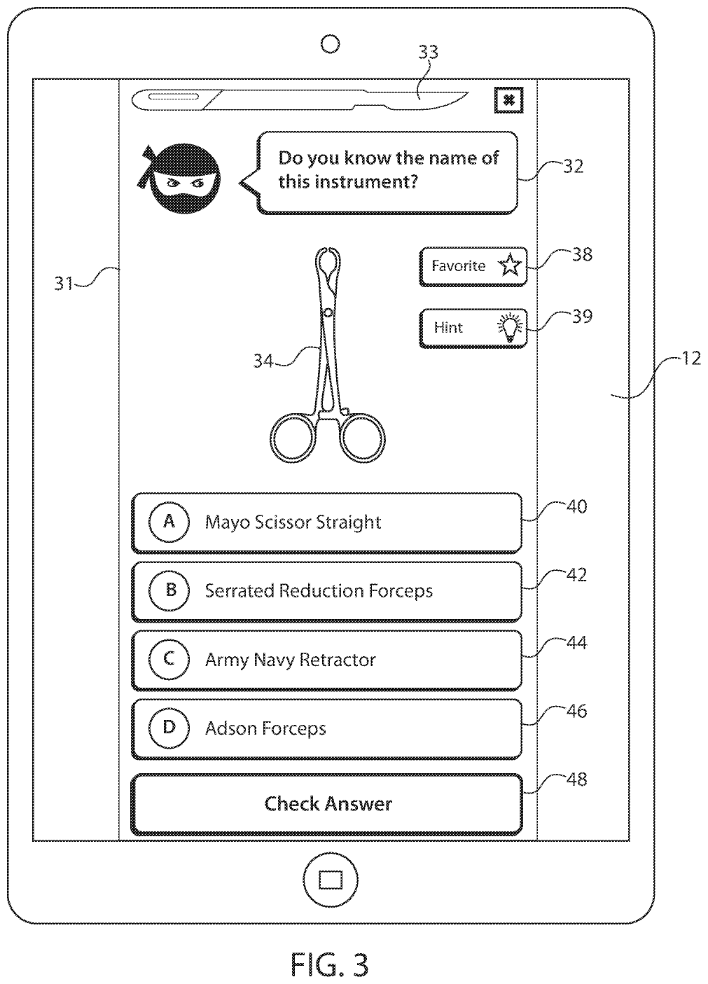

FIG. 3 illustrates a screenshot depicting a surgical instrument after the user selects a category of surgical instruments in FIG. 2, where the user is prompted to identify the depicted surgical instrument, in accordance with an embodiment of the present invention.

The user is presented with an image 34 of a surgical instrument and a question or query 32 in a flashcard 31 (electronic or digital flashcard) displayed on display screen 12. The question or query 32 posed is: Do you know the name of this instrument? Thus, the user needs to identify the surgical instrument shown by name.

In some embodiments, the educational software for displaying and providing access to learning surgical instrumentation in a guided environment displays the name of an associated lesson, learning unit, level of study, or instructional plan. In some embodiments, the surgical instrumentation training platform displays, by way of non-limiting examples, the names of learning activities, the topics of learning activities, and the types of learning activities. The learning activities relate to identifying surgical instrumentation and positioning surgical instrumentation on appropriate surgical trays. In some embodiments, the surgical instrumentation training platform displays one or more surgical images 34 associated with each learning activity. In further embodiments, a surgical image 34 is a photograph, screen capture, video, illustration, or animation. In some embodiments, the display 12 is organized to communicate a relationship between the learning activities. In further embodiments, the display 12 is organized to communicate a sequence, hierarchy, grouping, relative complexity, or relative difficulty.

In some embodiments, learning surgical instrumentation include visual and audio content, for example, text, images, video, audio, motion, interactivity, and animation. In further embodiments, interactive content allows a learner to interact with the learning activity through text, hyperlinks, graphical icons and other visual elements, manipulation of graphical elements, auditory elements, and motion elements. In view of the disclosure provided herein, surgical images 34 for learning surgical instrumentation are created by techniques known to those of skill in the art using machines, software, and languages known in the art.

It is noted that a double tap action can “expand” the image. Once expanded, the image can be further magnified with a pinch to zoom action. Additional images can be viewed by swiping right or left. Double tapping again demagnifies the image to restore the normal view.

The user can select a “favorite” button 38. The “favorite” button 38 allows the user to place the flashcard 31 in a custom stack that is accessible via, e.g., the app's home screen. The “favorite” button 38 allows the user to have quick access to instruments that require repeat practice. For instance, these may be flashcards the user has struggled with in the past. This feature saves time for the user as the user need not navigate through several subcategories to find the desired flashcard to review. Instead, all the flashcards are saved in, e.g., a “favorites” folder.

Also, each question related to a surgical instrument can be presented on a separate flashcard. Thus, a plurality of electronic flashcards can be used to present the surgical instrument questions to a user.

The user can be presented with, e.g., four options. Option A (40) can be: Mayo Scissor Straight, Option B (42) can be: Serrated Reduction Forceps, Option C (44) can be: Army Navy Retractor, and Option D (46) can be: Adson Forceps. A “Check Answer” button 48 can also be provided.

Regarding the generation of question 32, a question generator can be employed. The question generator can be implemented by artificial intelligence (AI), such as an artificial neural network (ANN). An ANN is a computational model composed of neurons (or simply nodes) and connections between the nodes. The strength of each connection is expressed by a numerical value called a “weight,” which can be modified. The activation of a given node is based on the activations of the nodes that have connections directed at that node and the weights on those connections. In general, a neural network incorporates some special nodes called “input nodes” with their activation external set, while other nodes are distinguished as “output nodes.”

As will be referred to herein, the word “question” and “query,” and their extensions, are used interchangeably and refer to the same concept, namely request for information. Such requests are usually expressed in an interrogative sentence, but they can also be expressed in other forms, for example as a declarative sentence providing a description of an entity of interest (where the request for the identification of the entity can be inferred from the context). “Structured information” (from “structured information sources”) is defined herein as information whose intended meaning is unambiguous and explicitly represented in the structure or format of the data (e.g., a database table). “Unstructured information” (from “unstructured information sources”) is defined herein as information whose intended meaning is only implied by its content (e.g., a natural language document). By “Semi structured” it is meant data having some of the meaning explicitly represented in the format of the data, for example a portion of the document can be tagged as a “title.”

In the exemplary embodiments, the ANN can be a system that generates the answer choices to questions. This will allow for the immersive experience to initially teach by process of elimination. Then, as time passes and expertise is gained, the questions will become more complicated such that the answers will be groups of similar category instruments (eliminating the possibility of process of elimination).

For example, when a participant is first exposed to a new instrument, the participant will be able to choose the instrument by the process of elimination, that is, “this is the only scissor, so it must be this.” As the game progresses, the participant will have to choose the instrument based on true knowledge and the other answers will all be other types of similar instruments (all the other answer choices will be scissors).

There will be, e.g., four levels of questions participants will need to complete:

Level 1: process of elimination—the only scissor in the answer choices.

Level 2: choose from 2 scissors and 2 non-scissors.

Level 3: choose from 3 scissors and one non-scissor.

Level 4: choose from 4 scissors.

Moreover, a tray challenge section is provided that will be unlocked upon completion of the appropriate sub-specialty level. Thus, once a participant completes, e.g., “urology” the participant can then go and practice building “urology trays” anytime they wish without going through the entire level.

In the scorecard section, the user or participant will be prompted to add wrong questions to the favorites to encourage them to practice the ones they struggle with.

In the count sheet game, the count sheet will be able to be viewed in its entirety via a zoom mode help button on the tray loading game so that the user can see all the instruments required to assemble a tray if they wish.

At the end of each level, there can be a cumulative test to make sure that the user is proficient in all items in each level. In one instance, after every 3 levels are completed, testing on all the items in the prior three levels can occur. One skilled in the art can contemplate a plurality of different testing mechanisms to keep participants skills up-to-date.

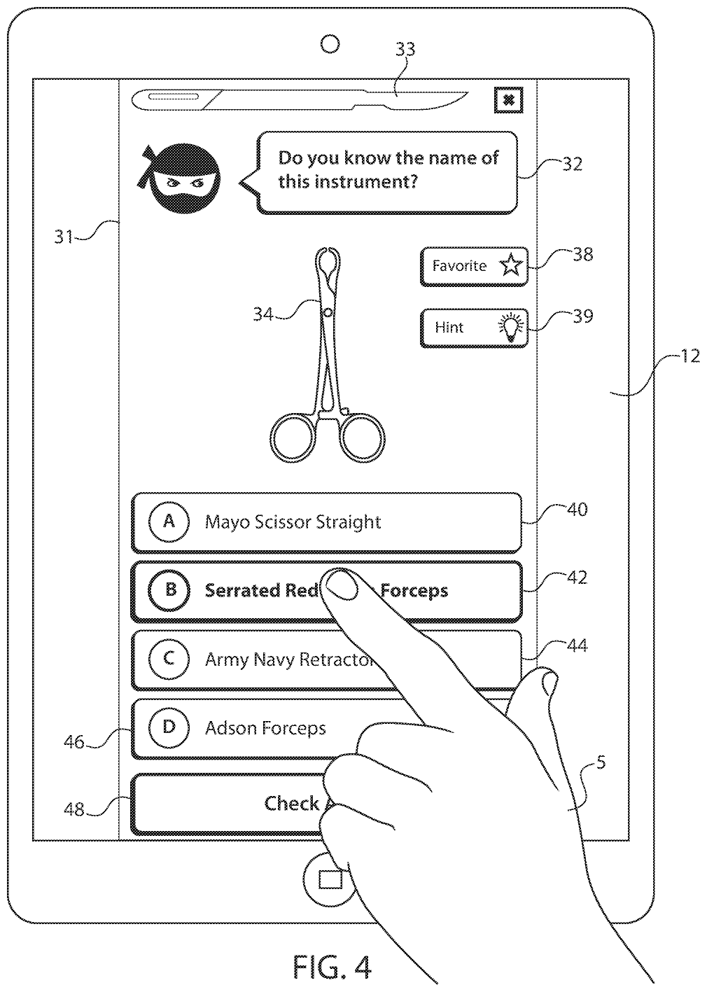

FIG. 4 illustrates a screenshot where the user selects an answer to the question posed in FIG. 3, in accordance with an embodiment of the present invention.

The user 5 decides that the correct answer for the displayed instrument 34 is Option B (42). Thus, the user 5 selects Option B (42) as the correct answer.

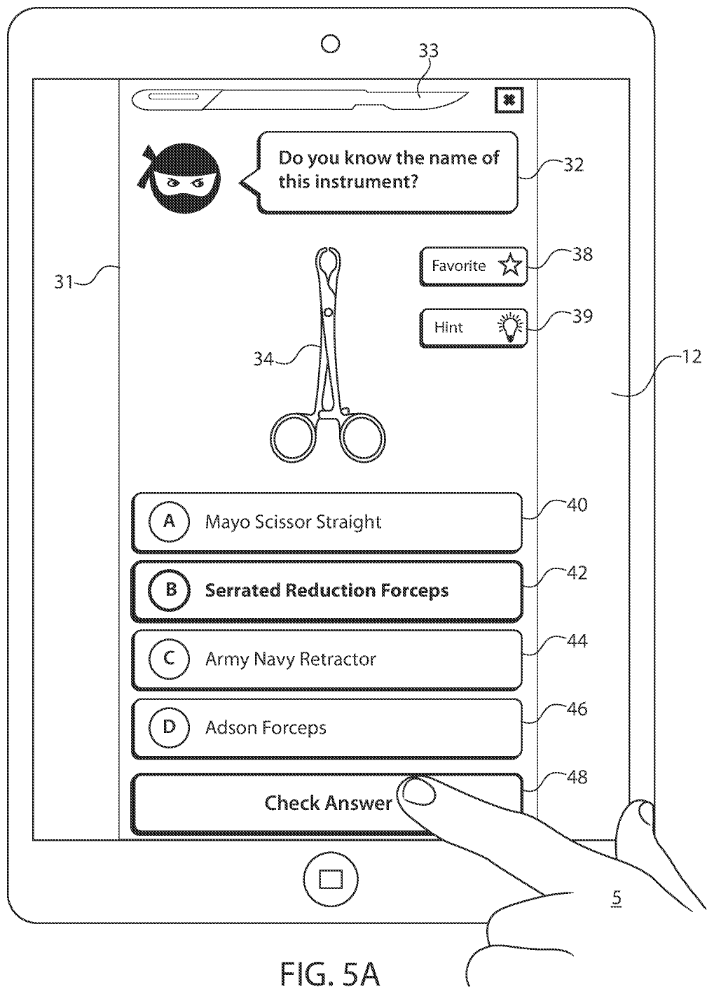

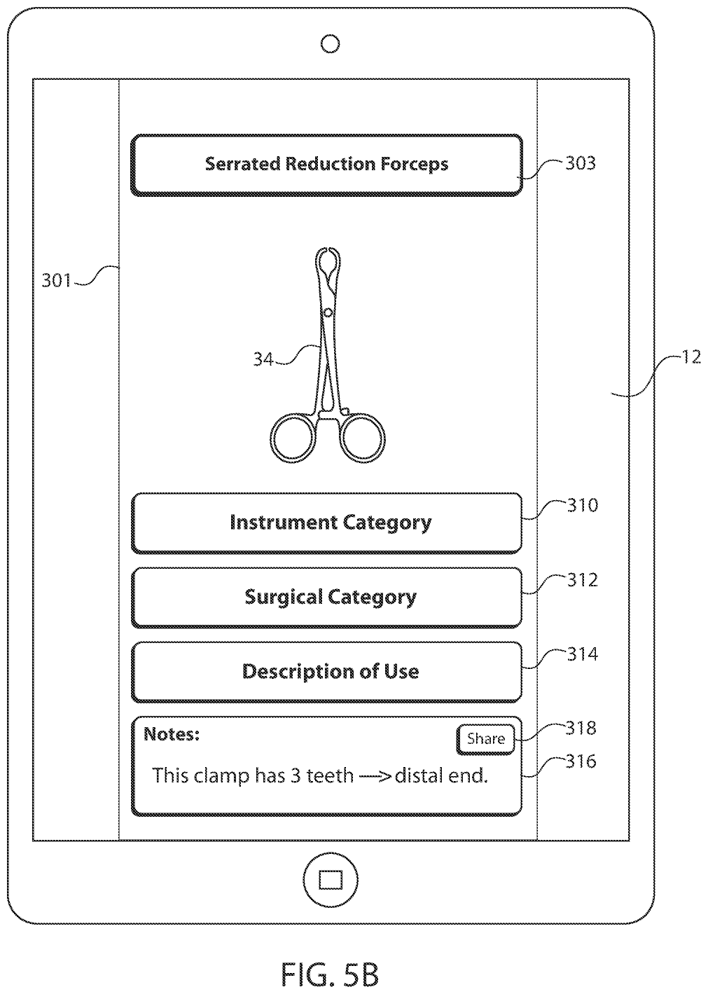

FIG. 5A illustrates a screenshot where the user checks the answer provided to the question posed, in accordance with an embodiment of the present invention.

The user 5 can instantly check the selected answer by pressing on the “Check Answer” button 48.

Of course, the user can be quizzed on more fields than just the name of the instrument. For example, the user can be quizzed with regards to the steps associated with its inspection and sterilization prior to use in surgery. Thus, one skilled in the art can contemplate quizzing the user on a variety of topics related to surgical instrumentation and surgical procedures.

FIG. 5B illustrates the back of the flashcard after the check answer button has been tapped, in accordance with an embodiment of the present invention.

After the check answer button 48 is tapped, the flashcard will “flip over” to display the backside 301 with the correct answer 303, an image of the instrument 34, and attributes associated with the instrument, such as its instrument category 310 (grasper, retractor, etc.) its surgical category 312 (general, urology, etc.), a brief description of its use in surgery 314, and a section 316 that allows a user to input custom text for learning reinforcement (a user's special individual notes 316 about the instrument [e.g., this clamp has three teeth on its distal end]). These custom notes may be sharable with other users via a “share” button 318. Other attributes can include other names the surgical instrument goes by. One skilled in the art can contemplate a plurality of different attributes related to the surgical instrument and to its use in surgical procedures.

A progress bar 33, in the shape of, e.g., a scalpel, can be displayed at a top portion of the flashcard 31 to indicate to the user how far along the user is in the current task.

In an alternative embodiment, hints can also be available in the form of 50/50, where one or two of the answer options are removed. The hint can be activated by a “Hint” button 39.

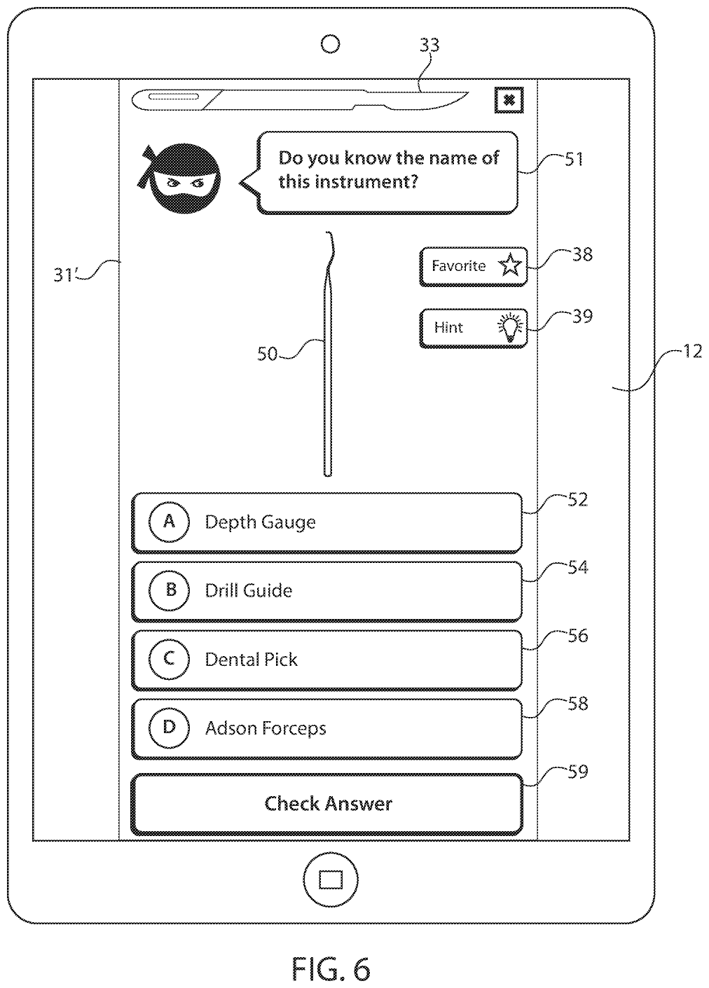

FIG. 6 illustrates a screenshot depicting another surgical instrument after the user identifies the previous surgical instrument, where the user is prompted to identify the depicted surgical instrument, in accordance with an embodiment of the present invention.

After the first surgical instrument has been identified in the sequence presented in FIGS. 1-5, another surgical instrument can be presented to the user. For example, the user is presented with an image 50 of an instrument and a question 51 in a flashcard 31′ displayed on display screen 12. The question 51 posed is: Do you know the name of this instrument? Thus, the user needs to identify the surgical instrument shown by name.

It is noted that a double tap action can “expand” the image. Once expanded, the image can be further magnified with a pinch to zoom action. Additional images can be viewed by swiping right or left. Double tapping again demagnifies the image to restore the normal view.

The user can select a “favorite” button 38. The “favorite” button 38 allows the user to place the flashcard 31 in a custom stack that is accessible via, e.g., the app's home screen. The “favorite” button 38 allows the user to have quick access to instruments that require repeat practice. For instance, these may be flashcards the user has struggled with in the past. This feature saves time for the user as the user need not navigate through several subcategories to find the desired flashcard to review. Instead, all the flashcards are saved in, e.g., a “favorites” folder.

The user can be presented with, e.g., four options. Option A (52) can be: Depth Gauge, Option B (54) can be: Drill Guide, Option C (56) can be: Dental Pick, and Option D (58) can be: Adson Forceps. A “Check Answer” button 59 can also be provided. The user can instantly check the selected answer by pressing on the “Check Answer” button 59.

A progress bar 33, in the shape of, e.g., a scalpel, can be displayed at a top portion of the flashcard 31′ to indicate to the user how far along the user is in the current task.

In an alternative embodiment, hints can also be available in the form of 50/50, where one or two of the answer options are removed. The hint can be activated by a “Hint” button 39.

This process can be repeated several or multiple times until a certain number of flashcards have been displayed to the user. In one instance, 10 flashcards are presented. In another instance, 20 flashcards are presented. In other instances the number of flashcards presented may be based on the number of correct answers provided by the user.

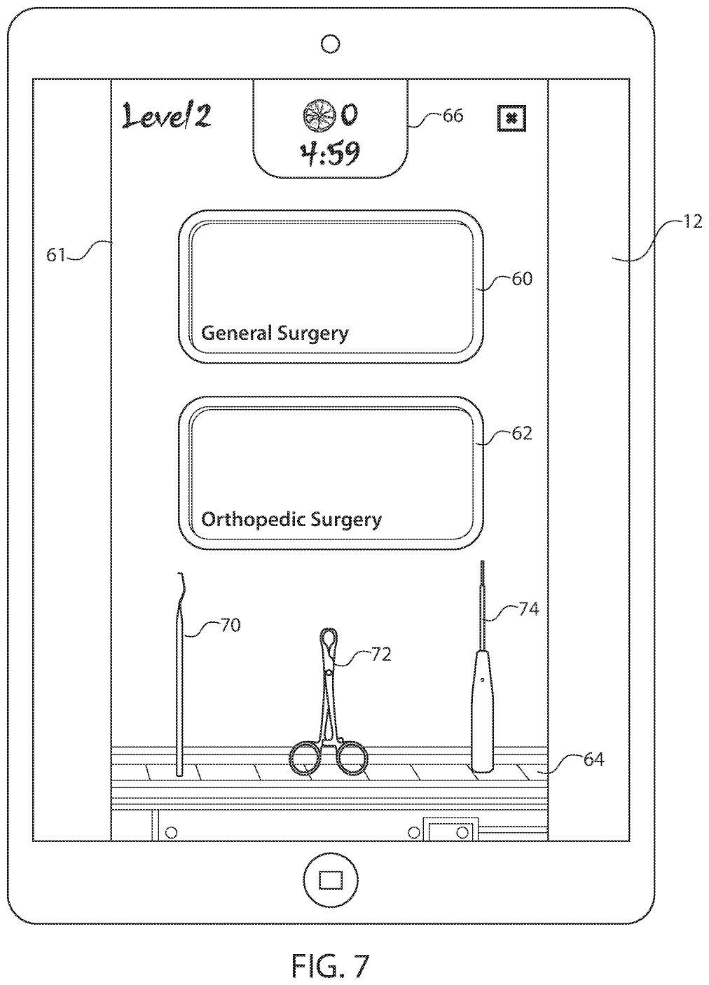

FIG. 7 illustrates a screenshot where the user has entered a second level after the first level has been successfully completed, the second level depicting surgical instruments on a conveyor belt and prompting the user to drag the surgical instruments into the correct surgical instrument category, in accordance with an embodiment of the present invention.

The flashcard 61 on display screen 12 illustrates a general surgery tray 60 and an orthopedic surgery tray 62. A conveyor belt 64 holds instruments 70, 72, 74. The user has a set amount of time 66 to place the instruments 70, 72, 74 onto the correct tray 60, 62.

Thus, the surgical instrumentation training platform can further challenge the user to categorize surgical instruments into subcategories and correct instrument type subcategories (e.g., grasper, retractor, etc.). The conveyer belt 64 on a bottom portion of the flashcard 61 can move from right to left and display surgical instruments 70, 72, 74. The user needs to drag the surgical instruments 70, 72, 74 into the correct subcategory as indicated on the upper half of the screen in the tray icons 60, 62.

The user can pause the conveyer belt 64 and double tap on an instrument to expand, pinch, and zoom the current image and scroll through its associated images. Double tapping the expanded instrument restores the normal conveyer belt display. Additionally, the speed of the conveyer belt 64 can be increased or decreased via a button displayed on the bottom, e.g., left corner of the screen 12.

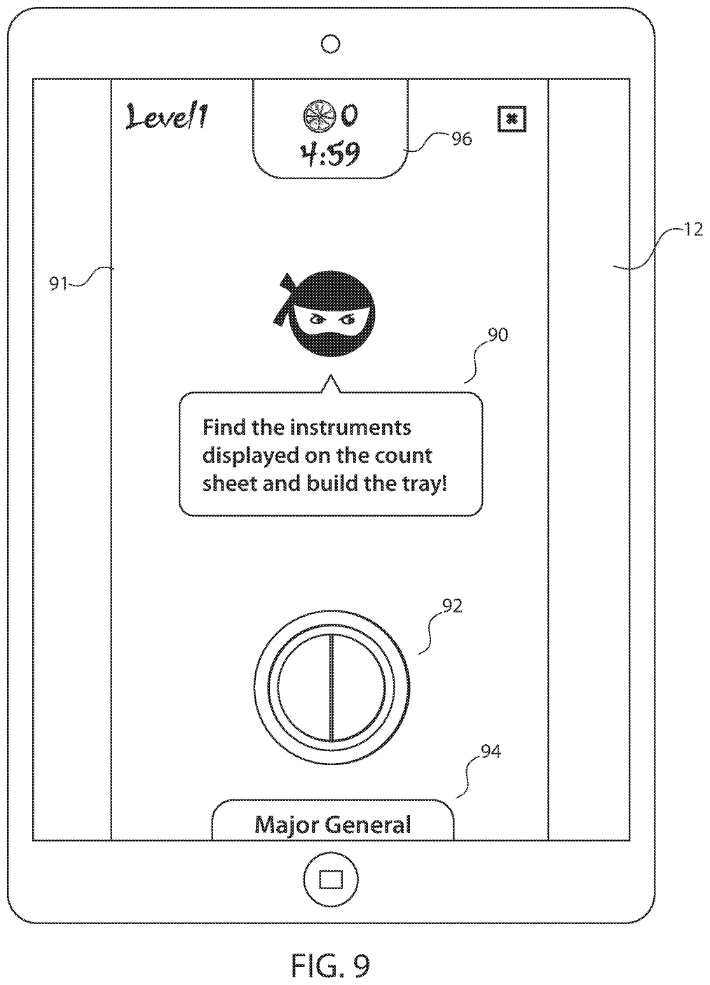

In this portion of the game, the user is challenged to assemble a “tray” of instruments that represents an actual tray used for surgery. Each tray has a unique combination of instruments which are listed on a “count sheet.” The object of this game is to find the instrument named at the top of the screen 12 among, e.g., a random jumble of instruments in a haphazard pile (FIGS. 9-12). The user drags the desired instrument into the tray icon on the bottom of the screen 12.

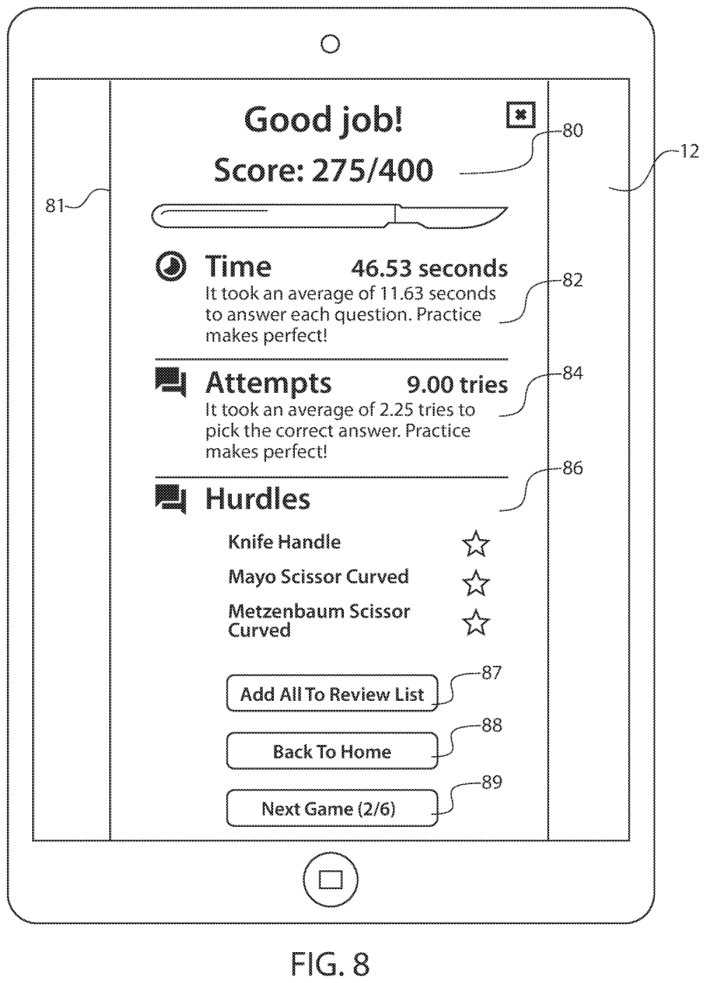

FIG. 8 illustrates a screenshot depicting a scorecard of the user, in accordance with an embodiment of the present invention.

Each challenge displays scoring and performance with indicators such as percentage correct, time spent on each question and task, standings among peers (a leaderboard), personal best, etc.

Flashcard 81 on screen 12 includes a scorecard displaying encouraging words and a score 80, a time 82 it took to the answer the questions, an average time it took to answer each question, an average number of attempts 84 it took to answer each question correctly, hurdles 86, an “Add All To Review List” button 87, a “Back To Home” button 88, and a “Next Game” button 89. The hurdles 86 can refer to difficult items or items that the user found challenging that can be added to a review list via the “Add All To Review List” button 88. Thus, the user can have control of which items to review at a future time.

Additionally, a user profile (e.g., accessed on the Home Screen) can have a calendar feature that allows a user to track the history of the game play with access to prior games played on prior dates and also has an icon in a calendar date indicating that a player played. Notifications can praise users for multi-day play (streaks) and also challenge them to continue or begin streaks.

Therefore, with respect to FIGS. 1-8, the user is shown a surgical instrument in a two or three-dimensional representation. The user can manipulate the surgical instrument on the screen in order to view it from multiple angles. The user is challenged to identify the surgical instrument. Once a user has mastered the identification of surgical instruments by name, the user is then challenged to categorize the surgical instruments into type of surgical instrument and then into type of surgical application. The user is shown each surgical instrument and then a variety of surgical categories, either represented by a list, an icon, an image, or another means.

FIG. 9 illustrates a screenshot where the user needs to assemble trays of instrumentation, in accordance with an embodiment of the present invention.

As noted, the second challenge that the user can face is to assemble trays with surgical instruments. The flashcard 91 of screen 12 displays a request 90, that is: Find the instruments displayed on the count sheet and build the tray. A tray can be designated as 92. The category can be designated as “Major General” 94. The user may have a specified amount of time 96 to complete the challenge. The user can click the screen 12 to proceed to view the surgical instruments.

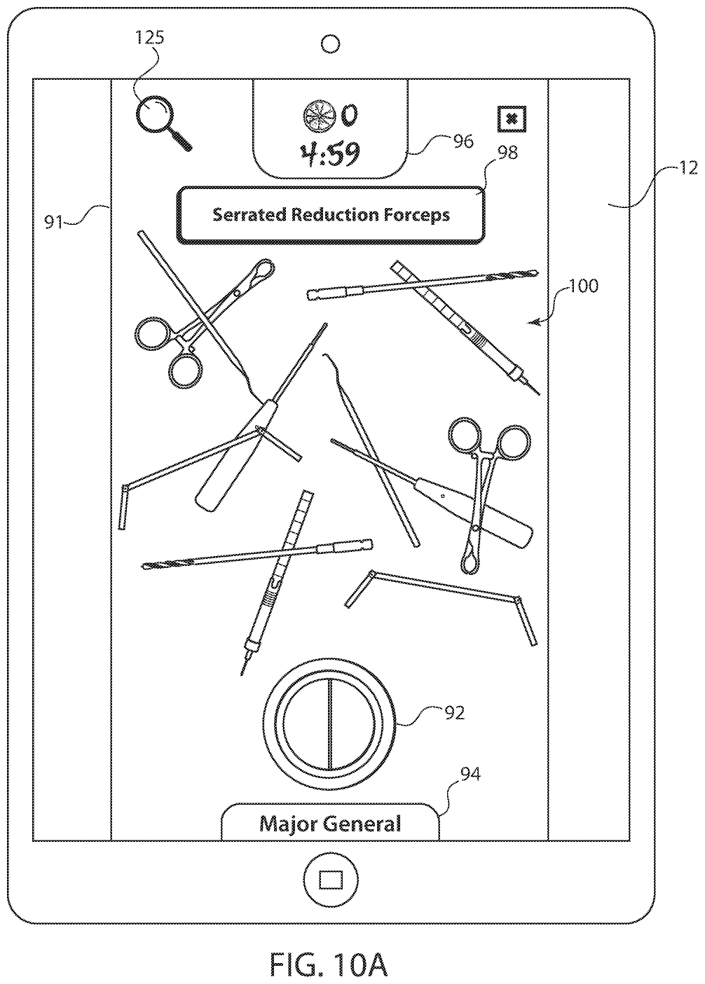

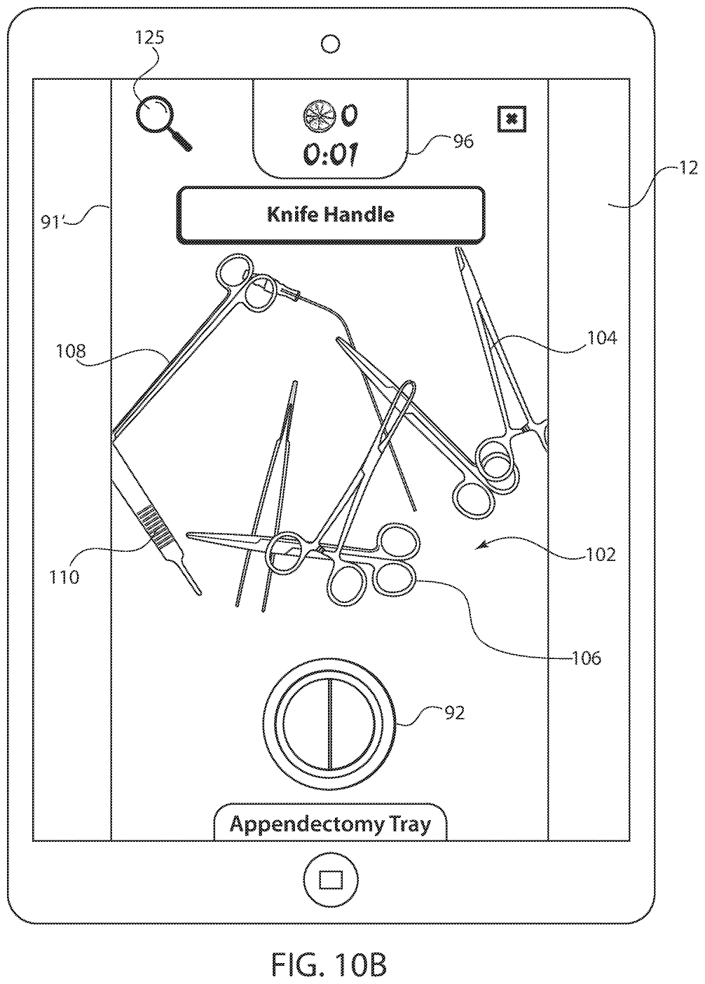

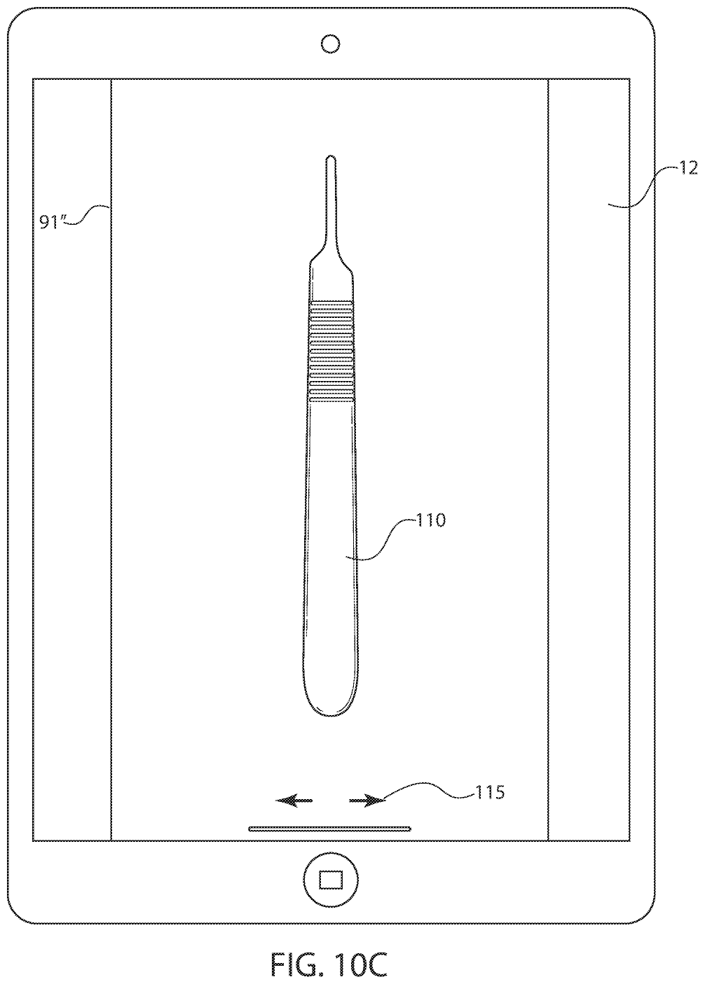

FIG. 10A illustrates a screenshot where a plurality of surgical instruments are presented and the user must assemble them into appropriate surgical categories, in accordance with an embodiment of the present invention.

Once the user proceeds, the flashcard 91 displays a plurality of surgical instruments 100. A name 98 of the surgical instrument can also be displayed. In the instant case, the name 98 of the surgical instrument selected is, e.g., “Serrated Reduction Forceps.” Thus, the name of the surgical instrument can be instantly displayed upon the user selecting such surgical instrument. The plurality of instruments 100 can be presented in a disorganized or jumbled manner as though thrown on a table. This allows for a more realistic or real-world experience where a tray or table includes a plurality of surgical instruments presented in a random manner.

Additionally, flashcard 91 can include a magnifying glass icon 125 that allows the user to magnify any of the plurality of instruments 100. In one example, the user can drag any of the plurality of instruments 100 over the magnifying glass icon 125 to enable magnification at various levels.

FIG. 10B illustrates a screenshot of further magnified views of surgical instruments of a surgical tray, in accordance with an embodiments of the present invention.

Further magnified views of surgical instruments 102 can be shown to the user. The magnifying icon 125 can provide the ability to view specific instruments, such as instruments 104, 106, 108, 110 on flashcard 91′. Flashcard 91′ can also include a timer 96. The further views can be viewed by the user swiping to the left or to the right.

FIG. 10C illustrates a screenshot of a single magnified instrument, in accordance with an embodiment of the present invention.

Flashcard 91″ illustrates the magnification of a single selected surgical instrument 110. The dot designations 115 can indicate that further magnified images of other surgical instruments are available. Thus, the user has the ability to magnify any of the surgical instruments on a surgical tray.

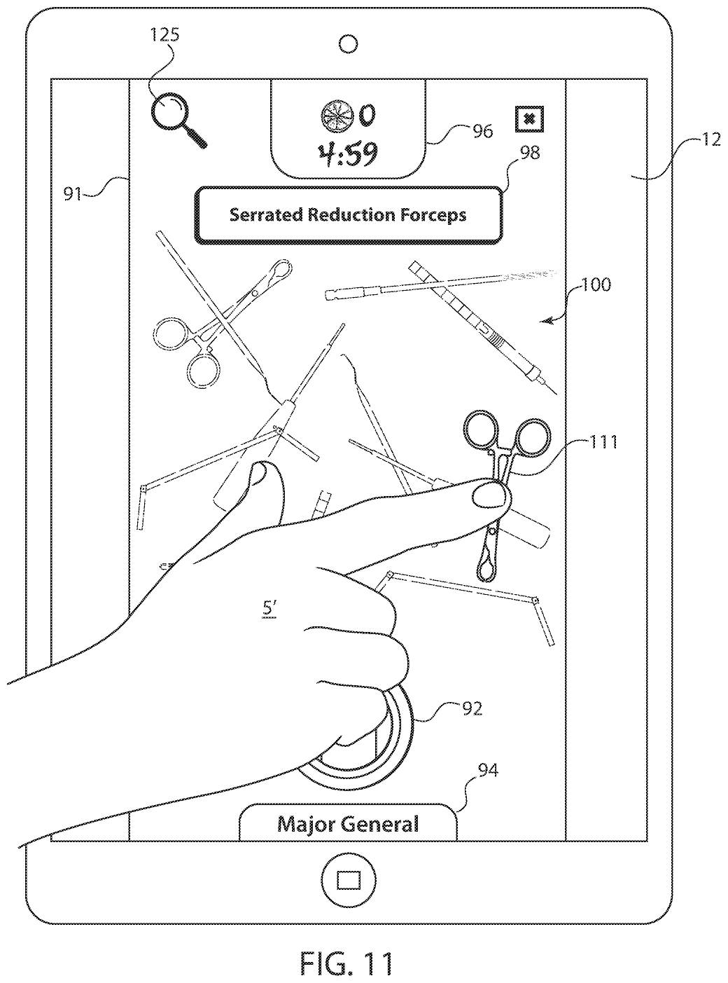

FIG. 11 illustrates a screenshot of the user selecting surgical instruments from the plurality of surgical instruments shown in FIG. 10A, in accordance with an embodiment of the present invention.

In the instant case, the user 5′ can select surgical instrument 111 with his/her finger.

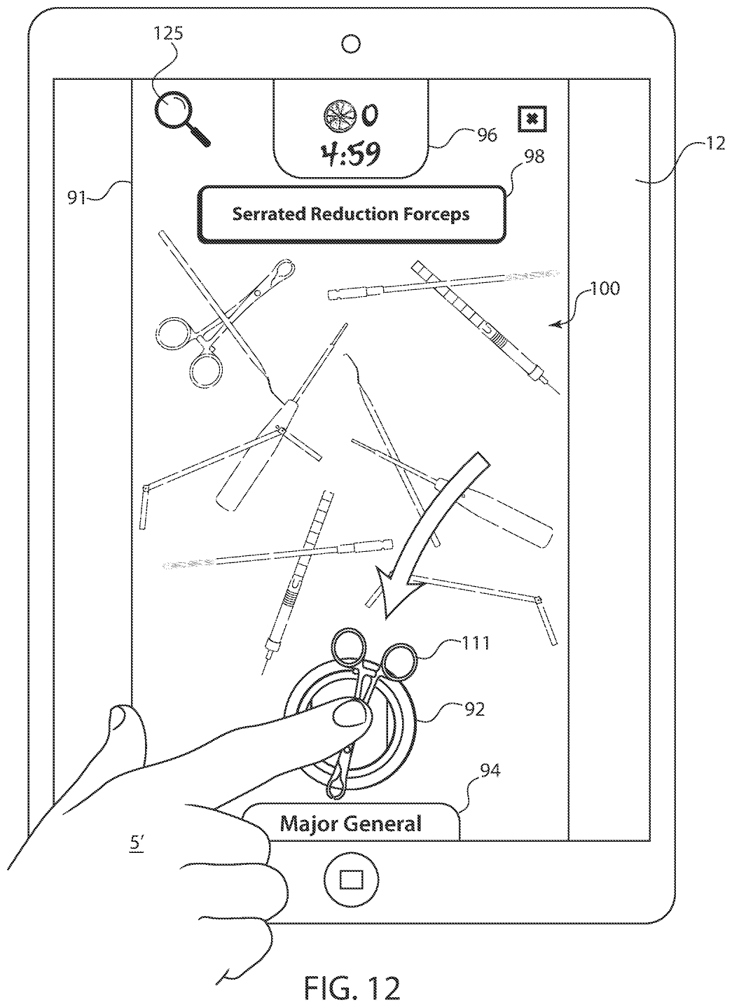

FIG. 12 illustrates a screenshot of the user dragging the selected surgical instruments onto a surgical instrument tray, in accordance with an embodiment of the present invention.

The user 5′ can then proceed to drag the surgical instrument 111 onto the tray 92. In other words, the user 5′ believes that surgical instrument 111 should be on a tray for such surgery. The user can then select further surgical instruments to be part of a tray for such specific surgical procedure.

Therefore, with respect to FIGS. 9-12, the user 5′ can be given a “count sheet.” A count sheet is a list of surgical instruments that are required to load a particular tray. The user 5′ will have to choose from various instruments 100, identify each instrument on the count sheet, and populate the tray 92 by either virtually placing it onto a stringer, virtually placing it into the tray, checking off a box on the list of the count sheet, or another means.

In another exemplary embodiment, the user 5′ is given an empty surgical tray with specific locations for each surgical instrument. The user is shown surgical instruments individually and is required to physically place them in their proper location on the surgical instrumentation tray.

In yet another exemplary embodiment, the user 5′ may be shown a surgical tray that is fully populated with surgical instruments and be asked to unload the surgical tray and organize it on a table. The table can represent the “back table,” which is the section of the surgical area upon which surgical instruments are laid out for use in a certain arrangement during surgery. These “back table” arrangements may be shared with other users and a central database to create a reference system for operating room personal to use for training new staff and to help current staff set up instrumentation for surgery.

In another embodiment, the user may also be shown groups of surgical instruments and be required to identify which surgical instrument does not belong in the same category as the others in the group of surgical instruments.

In yet another embodiment, the user may also be shown complex surgical instruments that require assembly and disassembly and be challenged to manipulate the components of the surgical instrument and assemble or disassemble such surgical instrument.

In another embodiment, the user can be challenged to complete the steps involved in the cleaning and inspection of instrument's for surgery consistent with the instruments “IFU-Instructions for Use.”

In another embodiment, an instrument lookup section can be provided that allows a user to search for an instrument by name and access the image of the instrument, as well information related to the instrument.

The surgical instrumentation training platform determines scores for each task completed. The scores will quantify, for example, the amount of time taken for each user to complete each task, the accuracy of each task performance, how often a user engages with each task, and how many times each user has engaged with a task. The surgical instrumentation training platform shows improvement or decline in proficiency over subsequent engagements and other data for teachers and administrators to use when analyzing a student's progress and proficiency.

In exemplary embodiments, each user can have a unique and secure username and password and be given the opportunity to create an icon for their profile. Administrators can have same. In some embodiments, the user signs up and creates an account with the system. As part of the sign up process, the user selects a unique username and password to access his/her account. The account creation and maintenance is performed independent of any institutions that the user might be affiliated with. For instance, when a user logins to his/her account with the surgical instrumentation training platform, the platform allows the user to access all surgical lessons/materials related to his/her past and current lessons. When the user moves from an institution to another one (from hospital to hospital or to other medical facilities), he/she can choose to maintain the same account and access privileges.

In other exemplary embodiments, a user may be given the opportunity to chat with other users during their use of the surgical instrumentation training platform. A user may be given the opportunity to challenge another user to head-to-head competition in completion of a certain task. There can be an opportunity for a multiplayer challenge so that many users can compete at the same time trying to complete a task related to surgical instrumentation.

Additionally, customization options, or “skins,” may employed by the user.

In yet another exemplary embodiment, a user can take notes regarding surgical instruments in order to augment their learning experience. By clicking or touching or some other means of interfacing with a specific instrument a “Notes” section would appear for a user to take notes for later reference. A user may be given an option to share these notes with other users.

The gamification of the surgical instrument selection allows for multiple paths of difficulty in the lessons to help students/learners find flow between boredom and anxiety. The educational gamification system creates a background failure environment for students/learners to try challenges, experiment, fail, and learn. Failure facilitates learning more efficiently than success. Exams and other graded activities typically focus on success and reward success. The goal of the gameful activities is to focus on re-do, on failure, and count units of progress. The gamification of the surgical instrumentation and surgical instrumentation trays achieves such purpose.

The educational gamification of the surgical instrumentation establishes autonomy and opens the possibility of the game to be enjoyable. This engenders emotion and opens up learning capacity. Participants collect points for completed tasks, which can be any set of surgical instrumentation activities created by the instructor. These surgical instrumentation activities can be likened to extra credit assignments with the difference in not counting directly towards a grade. Students who win the game do not automatically get a good grade in the class. While they do practice more, by completing surgical instrumentation tasks, it would be reasonable to believe that their grades would be positively affected by doing well in the game. Since the surgical instrumentation tasks are not directly related to grades, the pressure to complete the tasks under some penalty is much more relaxed than that of grades. The rewards in the educational gamification of the surgical instrumentation tasks can be promotions at work, free lunch, etc., not grades.

In some embodiments, the surgical instrumentation training platform for displaying and providing access to learning activities in a guided environment provides hyperlinks to learning activities to support the learner's use or demonstration of the activity in a guided environment. In some embodiments, the surgical instrumentation training platform provides access to full-length, full-feature learning activities related to surgical instrumentation and surgical instrumentation trays. In other embodiments, the surgical instrumentation training platform provides access to truncated or simplified activities. In some embodiments, a learner accesses a learning activity in a guided environment as part of live or recorded teaching. In further embodiments, a learner accesses a learning activity in a guided environment to model or demonstrate the activity to others.

The systems, platforms, servers, programs, and methods described herein further include surgical instrument education software for monitoring the progress of one or more learners. In some embodiments, the surgical instrumentation training platform for monitoring learner progress is adapted for use by a mentor/teacher to one or more learners. In further embodiments, the surgical instrumentation training platform is only accessible by a mentor/teacher. In various embodiments, the surgical instrumentation training platform for monitoring learner progress allows a mentor/teacher to monitor the progress of, for example, a single learner, a group of learners, or a class of learners. In further embodiments, the progress of a group or class of learners is monitored in aggregate. In other embodiments, the progress of a group or class of learners is monitored at the level of individual learners. In some embodiments, the surgical instrumentation training platform for monitoring learner progress is adapted for use by a learner. In further embodiments, the surgical instrumentation training platform is accessible by a mentor or a learner. In still further embodiments, the surgical instrumentation training platform for monitoring learner progress allows a learner to monitor their own progress. In still further embodiments, the surgical instrumentation training platform for monitoring learner progress allows a learner to compare their progress to the aggregate progress of a group or class of learners. In still further embodiments, the surgical instrumentation training platform for monitoring learner progress allows a learner to compare their progress to that expected by a mentor/teacher.

In some embodiments, the surgical instrumentation training platform for monitoring learner progress generates an on-screen progress report. In further embodiments, the surgical instrumentation training platform generates a printable progress report. In some embodiments, the surgical instrumentation training platform reports the names of each learning activity assigned. In some embodiments, the surgical instrumentation training platform reports the names of each learning activity completed. In some embodiments, the surgical instrumentation training platform for monitoring learner progress reports the number of learning activities completed. In further embodiments, the surgical instrumentation training platform reports the percentage of activities completed within a specific group of activities. In still further embodiments, the surgical instrumentation training platform reports the number of times each activity has been completed by a learner. In various embodiments, the surgical instrumentation training platform reports, for example, the date of assignment for each learning activity, the date of completion for each learning activity, the difficulty of each learning activity, and the number of days past due for each learning activity. In some embodiments, the surgical instrumentation training platform reports the educational objectives achieved. In further embodiments, the educational objectives achieved are reported in conjunction with the objectives of an instructional plan or the objectives in a published educational standard. In some embodiments, the surgical instrumentation training platform reports learner progress within an overall instructional plan or published educational standard. In some embodiments, the surgical instrumentation training platform for monitoring learner progress identifies learning activities assigned as independent work.

In light of the disclosure provided herein, those of skill in the art will recognize that in various embodiments a surgical instrumentation training platform for monitoring the progress of one or more learners is implemented in a multitude of ways. In some embodiments, the surgical instrumentation training platform for monitoring the progress of one or more learners is a feature integrated into one or more graphical user interfaces (GUIs). In further embodiments, the surgical instrumentation training platform is accessible by interacting with a link or icon integrated into a GUI. In some embodiments, the surgical instrumentation training platform for monitoring learner progress comprises a web browser plug-in, add-in, add-on, or extension. In further embodiments, a web browser, plug-in, add-in, add-on, or extension is accessible via, by way of non-limiting examples, click, right click, menu selection, keystroke, touchscreen gesture, or voice command.

In some embodiments, the surgical instrumentation training platform for displaying and providing access to learning activities generates an electronic communication for one or more learners, wherein said communication is selected from: email, web mail, short message service (SMS), multimedia messaging service (MMS), microblog post, social network post, instant message, or voice mail. In some embodiments, the surgical instrumentation training platform for displaying and providing access to learning activities allows sorting, organizing, or filtering surgical instrumentation activities by one or more of: assigning mentor/teacher, assignment date, due date, subject, and type of activity. In some embodiments, the system further includes a surgical instrumentation training platform for monitoring learner progress. In further embodiments, the surgical instrumentation training platform for monitoring learner progress identifies learning activities assigned as independent work and allows monitoring of independent activities separately from other activities. In further embodiments, the surgical instrumentation training platform for monitoring learner progress generates a printable report.

In some embodiments, the learning activities are organized according to an instructional plan designed to accomplish one or more educational objectives in at least one area of skill, interest, or expertise through the mutual reinforcement of individual activities. In further embodiments, an instructional plan interconnects two or more activities associated with a surgical subject in such a way that understanding is built progressively with one or more activities adding to the educational messages of one or more previous activities. In some embodiments, the surgical instrumentation training platform is adapted for use by a mentor/teacher and further adapted to support a mentor's/teacher's implementation of an instructional plan. In further embodiments, the surgical instrumentation training platform is only available to a person identified by the lesson delivery server as a mentor/teacher. In other embodiments, access to the surgical instrumentation training platform is not restricted.

The systems, platforms, servers, programs, and methods described herein include a surgical instrumentation training platform for assigning one or more learning activities as independent work to one or more learners. Independent work is work suggested or assigned by a mentor/teacher to be attempted or performed by one or more learners without continuous guidance. Independent work, as used herein, can include homework or work performed in a guided environment where a mentor is present, but not continuously providing guidance. In some cases, independent work is to be attempted or performed by individual learners. In other cases, independent work is to be attempted or performed by groups of learners in collaboration. In some embodiments, the surgical instrumentation training platform is adapted for use by a mentor/teacher and further adapted to support a mentor's/teacher's implementation of an instructional plan. In further embodiments, the surgical instrumentation training platform is only available to a person identified by the lesson delivery server as a mentor/teacher.

In some embodiments, the surgical instrumentation training platform for assigning learning activities as independent work allows a mentor/teacher to preview learning activities to learners in a guided environment prior to assignment of the activities as independent work. In further embodiments, the preview is of a truncated or simplified version of the activity. In other embodiments, the preview is of a full-length, full-featured version of the activity.

In further embodiments, a mentor/teacher assigns a learning activity as independent work for, by way of non-limiting examples, a single learner, group of learners, and a class of learners. In various embodiments, the surgical instrumentation training platform allows a mentor/teacher to configure additional parameters for each learning activity assigned as independent work including, by way of non-limiting examples, due date, overdue date, point value, learner notifications, and the amount of time the independent work is available to surgical instrumentation training platform for displaying and providing access to learning activities assigned as independent work.

The surgical instrumentation training platform for assigning one or more learning activities as independent work to one or more learners is implemented, in part or in whole, as part of a web application, a mobile application, a standalone application, or an extension to a web browser application.

The surgical instrumentation training platform maintains a history of student and instructor users' activities. For student users this history log includes the following information related to all past and current enrolled activities: surgical instrument lessons, rankings, reviews, and feedback by instructors. For instructor users this history log includes the following information related to all past and current offered activities: surgical instrument lessons, student reviews, assessments, lesson outcome, and ratings of individual lesson notes. Users have full control on accessibility of their history information elements.

Some embodiments of the surgical instrumentation training platform provide data-mining and statistical analysis tools to apply on information elements related to a set of student and instructor users. Data-mining and statistical analysis tools cover features such as: analyzing and categorizing users' profiles in terms of surgical instrument lessons taken, technical expertise, skills, rankings, potential future interests in surgical instrument lessons and technical-notes, network of linked users, professional and educational trends of other users with common technical profiles, data-mining of reviews, assessments, ratings of all surgical materials and instructors by other users to extract patterns and trends, and data-mining and pattern extraction of educational and professional moves by users of similar background/profile.

Some embodiments of the surgical instrumentation training platform provide suggestions to users (students or instructors) on potential helpful surgical materials that may help/interest them based on analyzing their educational profiles and history.

Some embodiments of the surgical instrumentation training platform provide job requirement matching capability based on users' professional and educational profiles and history. This matching includes: associating job posting requirements with technical background/expertise in terms of surgical lessons taken, level of success in certain surgical lessons, evaluations/assessments, associating job posting requirements with level of success in certain independent examination tests administered, and associating a job requirement matching based on similarity and correlation of a user's technical and professional background against those of some “reference” users employed within the same or different hospitals or medical facilities.

Some embodiments of the surgical instrumentation training platform allow an employer (e.g., hospital) to create detailed job posting requirements based on surgical lessons taken, rankings, assessment by certain instructors, and desired technical skills. The employers provide feedback to the surgical instrumentation training platform on their assessment of their past hires through the disclosed system (level of satisfaction) to enable the surgical instrumentation training platform to improve future matches or suggestions (based on results compiled from the surgical instrumentation gamification system). The disclosed system can analyze job postings and trends by employers (and their technical/surgical requirements) to provide feedback to student users to adjust and optimize their educational direction and profile (e.g., to suggest certain surgical lessons, etc.). This enables students to customize their surgical lessons to potential job postings. Hyperlinks to job postings may be available and users can potentially apply for jobs via the app.

Social and professional networking among all user types can be provided. Features and capabilities can be designed and customized considering different usage scenarios of the disclosed system. The tasks include: search capabilities for screening or finding users based on their profile/history or commonality between educational profiles, networking among the participants of a surgical lesson (e.g., students and the instructors), networking capability among student users enrolled in similar surgical lessons offered by different institutions or hospitals or medical facilities, and professional networking and interaction based on profiles/histories and/or commonalities between educational profiles (and surgical lessons taken).

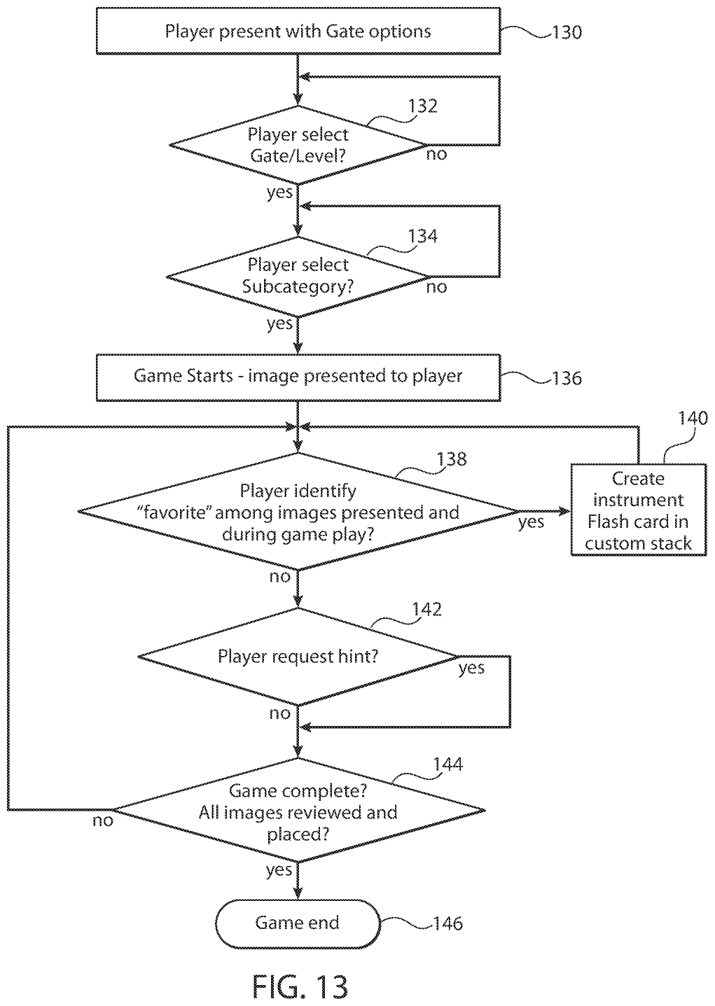

FIG. 13 illustrates a flowchart of how the user identifies, characterizes, and organizes surgical instrumentation and surgical instrumental trays of the surgical instrumentation training platform, in accordance with an embodiment of the present invention.

At block 130, a user is presented with gate options. The gate options represent different playing levels.

At block 132, it is determined whether the user has selected a gate or level. If NO, the process goes back to block 130. If YES, the process proceeds to block 134.

At block 134, it is determined whether the user has selected a sub-category. If NO, the process goes back to block 132. If YES, the process proceeds to block 136.

At block 136, the educational software commences. The training program commences by presenting an image of a surgical instrument to the user.

At block 138, it is determined whether the user pressed the “favorite” button during play. If YES, a surgical instrument flashcard is created in a custom stack 140 and saved in a “favorites” folder. If NO, the process proceeds to block 142.

At block 142, it is determined whether the user pressed the “hint” button during play. If YES, certain options are removed. If NO, the user selects a surgical instrument.

At block 144, it is determined whether the game is complete and whether all the surgical instrument images have been reviewed (in all the available flashcards). If YES, the game ends at 146. If NO, the play continues at block 148.

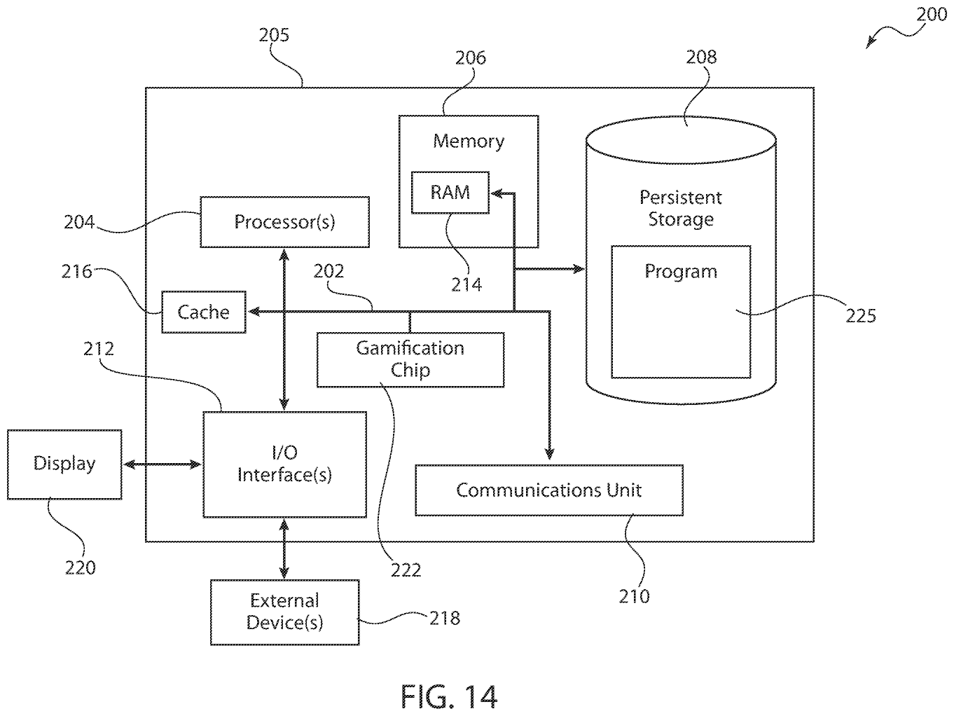

FIG. 14 is a block/flow diagram of an exemplary processing system employed in a surgical instrument gamification environment, in accordance with an embodiment of the present invention.

FIG. 14 depicts a block diagram of components of system 200, which includes computing device 205. It should be appreciated that FIG. 14 provides only an illustration of one implementation and does not imply any limitations with regard to the environments in which different embodiments can be implemented. Many modifications to the depicted environment can be made.

Computing device 205 includes communications fabric 202, which provides communications between computer processor(s) 204, memory 206, persistent storage 208, communications unit 210, and input/output (I/O) interface(s) 212. Communications fabric 202 can be implemented with any architecture designed for passing data and/or control information between processors (such as microprocessors, communications and network processors, etc.), system memory, peripheral devices, and any other hardware components within a system. For example, communications fabric 202 can be implemented with one or more buses.

Memory 206, cache memory 216, and persistent storage 208 are computer readable storage media. In this embodiment, memory 206 includes random access memory (RAM) 214. In another embodiment, the memory 206 can be flash memory. In general, memory 206 can include any suitable volatile or non-volatile computer readable storage media.

In some embodiments of the present invention, deep learning program 225 is included and operated by a surgical instrument gamification chip 222 as a component of computing device 205. In other embodiments, deep learning program 225 is stored in persistent storage 208 for execution by surgical instrument gamification chip 222 in conjunction with one or more of the respective computer processors 204 via one or more memories of memory 206. In this embodiment, persistent storage 208 includes a magnetic hard disk drive. Alternatively, or in addition to a magnetic hard disk drive, persistent storage 208 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 208 can also be removable. For example, a removable hard drive can be used for persistent storage 208. Other examples include optical and magnetic disks, thumb drives, and smart cards that are inserted into a drive for transfer onto another computer readable storage medium that is also part of persistent storage 208.

Communications unit 210, in these examples, provides for communications with other data processing systems or devices, including resources of distributed data processing environment. In these examples, communications unit 210 includes one or more network interface cards. Communications unit 210 can provide communications through the use of either or both physical and wireless communications links. Deep learning program 225 can be downloaded to persistent storage 208 through communications unit 210.

I/O interface(s) 212 allows for input and output of data with other devices that can be connected to computing system 200. For example, I/O interface 212 can provide a connection to external devices 218 such as a keyboard, keypad, a touch screen, and/or some other suitable input device. External devices 218 can also include portable computer readable storage media such as, for example, thumb drives, portable optical or magnetic disks, and memory cards.

Display 220 provides a mechanism to display data to a user and can be, for example, a computer monitor.

The systems, platforms, servers, programs, and methods disclosed herein include a processing system 200 that is optionally connected to a computer network. A computer network is a collection of computers and/or devices interconnected by communications channels that facilitate communications among users and allow users to share resources. In view of the disclosure provided herein, the computer network is created by techniques known to those of skill in the art using hardware, firmware, and software known to the art. In some embodiments, the computer network is a private network such as an intranet. In some embodiments, the computer network is the Internet. In further embodiments, the Internet provides access to the World Wide Web and the computer program and/or mobile application is provided to the digital processing device via the Web. In still further embodiments, the Internet provides access to the World Wide Web and the computer program and/or mobile application is provided to the digital processing device via cloud computing. In other embodiments, the computer network comprises data storage devices including, by way of non-limiting examples, CD-ROMs, DVDs, flash memory devices, solid state memory, magnetic disk drives, magnetic tape drives, optical disk drives, cloud computing systems and services, and the like. In further embodiments, the computer program and/or mobile application is provided to the digital processing device via a data storage device.

In summary, regarding FIGS. 1-14, the two main challenges that the sterile processing department of a hospital faces are instrument recognition and tray accuracy. The surgical instrumentation training platform as described in this invention aims to minimize error associated with improperly identifying instruments and improperly loading a surgical tray for surgery that could result in missing instruments during an operation. Additionally, surgical support staff such as Scrub Techs need to have precise knowledge of surgical instrumentation and would benefit from the exemplary surgical instrumentation training platform. The main challenge that surgical support staff such as a Scrub Tech face is similar to the Sterile Processing Technicians that assemble trays. They also need to be able to accurately identify a surgical instrument as well as its proper location in a surgical tray. The surgical instrumentation training platform as described in the exemplary invention improves the Scrub Tech's knowledge of surgical instrumentation in order to minimize surgical error and improve surgical efficiency. It also provides a way to practice setting up for surgery by allowing students/learners to unpack surgical instrument trays and place their contents on a simulated surgical table in a desired arrangement.

Additionally, instrument manufacturing companies utilize sales representatives to interface with surgeons and hospital support staff. These representatives need to become subject matter experts in order to properly assist staff. Accordingly, sales representatives would also benefit from the exemplary learning platform. Sales representatives need to learn the names of instruments as well as their proper locations in a surgical tray. Additionally, sales representatives need to know the proper sequence of use in surgery, which could be taught via a “what's next?” type game which can be created by simple modification of the count sheet game. Furthermore, Scrub Techs need to know the proper sequence of instrument usage as well, and thus would benefit from the aforementioned game.

Gamification elements such as points, badges, streaks, leaderboards, and leveling-up are incorporated into the surgical instrumentation platform.

A user can earn points by completing tasks. The reward may exist in the form of in-game currency. The currency may be redeemed for items of real value or for use in the game to improve the student's experience.

Badges can be used in order to display competence achievements in a variety of subspecialties in the surgical domain. Badges serve as a form of micro-credentialing and proof of knowledge and competency. Employers may choose to use these micro-credentials as a way to screen applicants for desired positions.

Streaks can be used to incentivize a user to engage with the surgical instrumentation platform on a regular basis and such action may be rewarded with points or badges or other types of rewards (e.g., free lunch, work promotions, etc.).

Leaderboards can show a user's standing against his or her peers. For example, each user's accuracy and time to completion scores for tests and tasks can be displayed on the leaderboard for comparison and incentive. The concept of leveling-up can be used in order to gradually impart more complex knowledge to the user. The user can start, for example, with the basics of surgical instrument identification and pass through a series of graduated levels until the user achieves mastery as deemed by the instructors. Mastery may come in the form of being able to accurately load a surgical tray, for example. These gaming elements serve to increase engagement with the surgical instrumentation teaching and training platform.

Preferably, the surgical instruments are selected from a group consisting of: needle holders, needle drivers, graspers, forceps, vessel sealing devices, dissectors, resectors, probes, morcellators, ultrasonic instruments, video-assisted devices, clip appliers, surgical staplers, coagulators, bipolar an mechanical scissors, irrigation instruments, and suction instruments.

In another embodiment, the plurality of surgical instruments is selected from a group consisting of: vessel sealers, coagulators, biopsy instruments, needles, probes, sensors, graspers, forceps, knives, scissors, sutures, balloon dissectors, stents, irrigators, suction devices, stabilizers, blunt dissectors, lasers, optical devices, implants and anchors.

Other surgical instruments and devices that may be utilized include: biopsy instruments, sensors, graspers, sutures, stents, irrigators, balloon dissectors, suction devices, stabilizers, blunt dissectors, lasers, optical devices, anchors, tissue ablators, etc.

The systems, platforms, servers, programs, and methods described herein include a plurality of learning activities organized according to an instructional plan designed to accomplish one or more educational objectives in at least one area of skill, interest, or expertise. An area of skill, interest, or expertise includes topics such as surgical instrumentation and surgical instrumentation trays.

In alternative embodiments, one or more areas of skill, interest, or expertise are selected from science topics including, by way of non-limiting examples, health, nutrition, biology, ecology, environmental science, meteorology, oceanography, marine biology, hydrology, soil science, botany, anatomy, physiology, virology, pharmacology, cell biology, molecular biology, microbiology, genetics, medicine, pathology, neuroscience, zoology, chemistry, organic chemistry, biochemistry, physical chemistry, materials science, biophysics, physics, optics, earth science, electrical engineering, civil engineering, mechanical engineering, chemical engineering, and computer science. All these science topics may include elements related to surgical instrumentation and surgical instrumentation trays, and in certain embodiments, may be incorporated into the flashcards of the surgical instrumentation teaching and training platform.

The present invention can be a system, a method, and/or a computer program product. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory, a read-only memory, an erasable programmable read-only memory (EPROM or Flash memory), a static random access memory, a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can include copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions can be provided to at least one processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks or modules. These computer readable program instructions can also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks or modules.

The computer readable program instructions can also be loaded onto a computer, other programmable data processing apparatus, or other device to cause a series of operational blocks/steps to be performed on the computer, other programmable apparatus or other device to produce a computer implemented process, such that the instructions which execute on the computer, other programmable apparatus, or other device implement the functions/acts specified in the flowchart and/or block diagram block or blocks or modules.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which includes one or more executable instructions for implementing the specified logical function(s). In some alternative implementations, the functions noted in the blocks can occur out of the order noted in the figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While there have been shown, described and pointed out fundamental novel features of the present principles, it will be understood that various omissions, substitutions and changes in the form and details of the methods described and devices illustrated, and in their operation, may be made by those skilled in the art without departing from the spirit of the same. For example, it is expressly intended that all combinations of those elements and/or method steps which perform substantially the same function in substantially the same way to achieve the same results are within the scope of the present principles. Moreover, it should be recognized that structures and/or elements and/or method steps shown and/or described in connection with any disclosed form or implementation of the present principles may be incorporated in any other disclosed, described or suggested form or implementation as a general matter of design choice. It is the intention, therefore, to be limited only as indicated by the scope of the claims appended hereto.

It should also be understood that the example embodiments disclosed and taught herein are susceptible to numerous and various modifications and alternative forms. Thus, the use of a singular term, such as, but not limited to, “a” and the like, is not intended as limiting of the number of items. Furthermore, the naming conventions for the various components, functions, parameters, thresholds, and other elements used herein are provided as examples, and can be given a different name or label. The use of the term “or” is not limited to exclusive “or” but can also mean “and/or”.

Having described preferred embodiments, which serve to illustrate various concepts, structures and techniques that are the subject of this patent, it will now become apparent to those of ordinary skill in the art that other embodiments incorporating these concepts, structures and techniques may be used. Additionally, elements of different embodiments described herein may be combined to form other embodiments not specifically set forth above.

Accordingly, it is submitted that that scope of the patent should not be limited to the described embodiments but rather should be limited only by the spirit and scope of the following claims.

1. A computer-implemented method for employing a surgical instrument educational platform to train medical personnel to identify, characterize, and organize surgical instrumentation and surgical instrumentation trays, the computer-implemented method comprising:
employing an artificial neural network (ANN) to display a limited and pertinent set of surgical instrumentation related information to a user in a specific, structured manner by performing a plurality of non-transitory instructions including:
selecting a level from one or more levels on a display of an electronic device;
selecting a surgical category from a plurality of surgical categories;
in response to selecting the surgical category, displaying, on an electronic flashcard, an image of a surgical instrument on the display of the electronic device and further displaying, on the electronic flashcard, a query generated by the ANN to prompt the user to identify the image of the surgical instrument and answer choices to the query generated by the ANN, the electronic flashcard constructed to have a front side and a back side featuring a summary of targeted information associated with the image of the surgical instrument, the summary of targeted information on the back side restrained to a correct answer to the query, attributes associated with the image of the surgical instrument, and a custom text input section used for allowing the user to input custom personal notes, the custom personal notes sharable with others by activating a share icon positioned on the back side of the electronic flashcard, wherein the user directly accesses the back side of the electronic flashcard only while the query generated by the ANN is displayed;
loading, via a processor, surgical instrument options to allow the user to match the image of the surgical instrument to one of the surgical instrument options;
prompting the user to select an option from the surgical instrument options; and
in response to the selected option by the user, outputting a result.

2. The computer-implemented method of claim 1, wherein, after the result is outputted, the user challenges other users to head-to-head competition in completing a task related to the surgical instrumentation.

3. The computer-implemented method of claim 1, wherein a progress of the user is monitored and a progress report is generated, the progress report integrated onto the display of the electronic device and accessed via an icon or hyperlink.

4. The computer-implemented method of claim 1, wherein the electronic flashcard includes a favorite icon that allows the user to save the electronic flashcard in a personalized folder.

5. The computer-implemented method of claim 1, wherein the electronic flashcard includes a hint icon that allows the user to automatically remove one or more of the or two of the surgical instrument options.

6. The computer-implemented method of claim 1, wherein gamification elements are incorporated into the surgical instrument educational platform, the gamification elements including at least points, badges, streaks, leaderboards, and leveling-up.

7. The computer-implemented method of claim 1, wherein a fully populated surgical instrumentation tray is presented, and the user is prompted to unload the surgical instrumentation tray and organize it on a back table of a surgical area to create shareable back table arrangements.

8. The computer-implemented method of claim 6, wherein the user earns points representing in-game currency redeemable for real-value items and the user earns badges indicating competence achievements in a variety of sub-specialties in a surgical domain.

9. The computer-implemented method of claim 6, wherein the user is trained to assemble the surgical instrumentation trays such that the user can determine where each of the surgical instrumentation belongs on the surgical instrumentation trays.

10. A computer-implemented method for employing a surgical instrument educational platform to train medical personnel to identify, characterize, and organize surgical instrumentation and surgical instrumentation trays, the computer-implemented method comprising:
employing an artificial neural network (ANN) to display a limited and pertinent set of surgical instrumentation related information to a user in a specific, structured manner by performing a plurality of non-transitory instructions including:
selecting a level from one or more levels on a display of an electronic device;
selecting a surgical category from a plurality of surgical categories;
in response to selecting the surgical category, displaying, on an electronic flashcard in a first level, an image of a surgical instrument on the display of the electronic device and further displaying, on the electronic flashcard, a query generated by the ANN to prompt the user to identify the image of the surgical instrument and answer choices to the query generated by the ANN, the electronic flashcard constructed to have a front side and a back side featuring a summary of targeted information associated with the image of the surgical instrument, the summary of targeted information on the back side restrained to a correct answer to the query, attributes associated with the image of the surgical instrument, and a custom text input section used for allowing the user to input custom personal notes, the custom personal notes sharable with others by activating a share icon positioned on the back side of the electronic flashcard, and displaying on another electronic flashcard, in a second level triggered after completion of the first level, a plurality of surgical instruments moving on a conveyor belt and at least one surgical instrumentation tray, wherein the user directly accesses the back side of the electronic flashcard only while the query generated by the ANN is displayed;
prompting the user to select and drag any of the plurality of surgical instruments moving on the conveyor belt onto the at least one surgical instrumentation tray; and
in response to positioning the plurality of surgical instruments moving on the conveyor belt onto the at least one surgical instrumentation tray, outputting a result.

11. The computer-implemented method of claim 10, wherein the user has a predetermined amount of time to select and drag any of the plurality of surgical instruments moving on the conveyor belt onto the at least one surgical instrumentation tray.

12. The computer-implemented method of claim 10, wherein gamification elements are incorporated into the surgical instrument educational platform, the gamification elements including at least points, badges, streaks, leaderboards, and leveling-up.

13. The computer-implemented method of claim 10, wherein a fully populated surgical instrumentation tray is presented, and the user is prompted to unload the surgical instrumentation tray and organize it on a back table of a surgical area to create shareable back table arrangements.

14. The computer-implemented method of claim 12, wherein the user earns points representing in-game currency redeemable for real-value items and the user earns badges indicating competence achievements in a variety of sub-specialties in a surgical domain.

15. The computer-implemented method of claim 10, wherein the user is trained to assemble the surgical instrumentation trays such that the user can determine where each of the surgical instrumentation belongs on the surgical instrumentation trays.

16. The computer-implemented method of claim 10, wherein the surgical instrument educational platform determines at least a score for each task completed, an amount of time taken for each task to be completed, accuracy of each task performance, how often the user engaged with each task, and how many times the user engaged with the task.

17. A computer-implemented method for employing a surgical instrument educational platform to train medical personnel to identify, characterize, and organize surgical instrumentation and surgical instrumentation trays, the computer-implemented method comprising:
employing an artificial neural network (ANN) to display a limited and pertinent set of surgical instrumentation related information to a user in a specific, structured manner by performing a plurality of non-transitory instructions including:
selecting a level from one or more levels on a display of an electronic device;
selecting a surgical category from a plurality of surgical categories;
in response to selecting the surgical category, displaying, on an electronic flashcard in a first level, an image of a surgical instrument on the display of the electronic device and further displaying, on the electronic flashcard, a query generated by the ANN to prompt the user to identify the image of the surgical instrument and answer choices to the query generated by the ANN, the electronic flashcard constructed to have a front side and a back side featuring a summary of targeted information associated with the image of the surgical instrument, the summary of targeted information on the back side restrained to a correct answer to the query, attributes associated with the image of the surgical instrument, and a custom text input section used for allowing the user to input custom personal notes, the custom personal notes sharable with others by activating a share icon positioned on the back side of the electronic flashcard, and displaying on another electronic flashcard, in a second level triggered after completion of the first level, concurrently all of a plurality of surgical instruments in a jumbled manner within another electronic flashcard and at least one surgical instrumentation tray, wherein the user directly accesses the back side of the electronic flashcard only while the query generated by the ANN is displayed;
prompting the user to select and drag at least one surgical instrument of the plurality of surgical instruments onto the at least one surgical instrumentation tray; and
in response to positioning the at least one surgical instrument of the plurality of surgical instruments onto the at least one surgical instrumentation tray, outputting a result.

18. The computer-implemented method of claim 17, wherein gamification elements are incorporated into the surgical instrument educational platform, the gamification elements including at least points, badges, streaks, leaderboards, and leveling-up.

19. The computer-implemented method of claim 18,
wherein the user earns points representing in-game currency redeemable for real-value items; and
wherein the user earns badges indicating competence achievements in a variety of sub-specialties in a surgical domain.

20. The computer-implemented method of claim 17, wherein the surgical instrument educational platform determines at least a score for each task completed, an amount of time taken for each task to be completed, accuracy of each task performance, how often the user engaged with each task, and how many times the user engaged with the task.
